(19) United States
(12) Patent Application Publication
(10) Pub. No.: US 2019/0358258 A1
(43) Pub. Date: Nov. 28, 2019

(54) METHODS FOR ENHANCING PROLIFERATION OF T REGULATORY CELLS

(71) Applicants: Robert J. Deans, Cambridge, MA (US); James Reading, London (GB); Samantha Stubblefield, Twinsburg, OH (US); Timothy Tree, London (GB)

(72) Inventors: Robert J. Deans, Cambridge, MA (US); James Reading, London (GB); Samantha Stubblefield, Twinsburg, OH (US); Timothy Tree, London (GB)

(21) Appl. No.: 15/767,085

(22) PCT Filed: Oct. 9, 2015
(86) PCT No.: PCT/US2015/054941

§ 371 Date: Jul. 31, 2018

(51) Int. Cl. A61K 35/17; C12N 5/0783; C12N 5/0775; C07K 16/18

(52) U.S. Cl. CPC A61K 35/17; C07K 16/18; C12N 5/0663; C12N 5/0637

(57) ABSTRACT

The invention is generally directed to a method of enhancing proliferation of T regulatory cells (Tregs) in vitro, comprising contacting Tregs with cells (I), or conditioned medium from the cells, in the presence of one or more Treg stimulation agents. The Treg stimulation agent(s) is present in an amount and for a time effective to stimulate proliferation of the Tregs. The cells (I) are present in an amount and for a time effective to enhance proliferation of the Tregs. The cells (I) are non-embryonic stem, non-germ cells characterized by one or more of the following: extended replication in culture and express markers of extended replication, express markers of pluripotentiality, and have broad differentiation potential, are not tumorigenic or transformed, and have a normal karyotype. The invention is also directed to methods for immune modulation using the proliferated Tregs, cell banks, drug discovery methods, populations, and compositions of the proliferated Tregs.

### FIELD OF THE INVENTION

The invention provides methods for enhancing proliferation of T regulatory cells (Tregs) for therapeutic immune modulation and other uses. The invention is generally directed to a method of enhancing proliferation of Tregs in vitro, the method comprising contacting Tregs with cells (I), or conditioned medium from the cells, in the presence of one or more Treg stimulation agents. The one or more Treg stimulation agents is present in an amount and for a time effective to stimulate proliferation of the Tregs. The cells (I) are present in an amount and for a time effective to enhance proliferation of the Tregs. The cells (I) are non-embryonic stem, non-germ cells that can be characterized by one or more of the following: extended replication in culture and express markers of extended replication, such as telomerase, express markers of pluripotentiality, and have broad differentiation potential, are not tumorigenic or transformed, and have a normal karyotype. The invention is also directed to methods for immune modulation using the proliferated Tregs. The invention is also directed to cell banks that can be used to provide the proliferated Tregs for administration to a subject. The invention is also directed to drug discovery methods. The invention is also directed to populations of the proliferated Tregs. The invention is also directed to compositions of the proliferated Tregs, such as in pharmaceutical compositions.

### SUMMARY OF THE INVENTION

A central goal in global healthcare remains the development of therapeutics with the ability to induce long-term immunological tolerance in the absence potentially toxic pharmacologic immunosuppression. Regulatory T cells (Tregs) are a naturally occurring population of lymphocytes that have evolved to maintain immune homeostasis and resolve inflammatory immune responses via a spectrum of cell-mediated and soluble mechanisms.

Loss of Tregs through genetic mutations affecting the signature Treg transcription factor FoxP3 lead to systemic autoimmunity in mice and man, while pre-clinical data has firmly established that adoptive transfer or in vivo generation of Treg can remedy autoimmunity, graft-versus-host disease (GVHD), and solid organ transplant rejection. These observations are consistent in humanized mouse models, where adoptive transfer of Tregs has facilitated cessation of immune suppression and protection from lethal T effector cell (Teff)-mediated tissue destruction or organ failure.

Animal models have indicated that large polyclonal Treg cell numbers are required to achieve in vivo efficacy. Given that Treg numbers in peripheral blood are low (1-10% CD4⁺ T cells), and that Tregs exhibit unfavorable ex vivo proliferative kinetics, obtaining sufficient yields for transfer is technically challenging.

The current consensus “gold standard” protocol for expansion of polyclonal Tregs involves FACS sorting or magnetic isolation of CD4+CD25highCD127low lymphocytes from peripheral blood, followed by 4 rounds (each 10-14 days) of stimulation with anti-CD3, anti-CD28 beads in the presence of high concentrations of IL-2 and the immunosuppressive drug rapamycin (included to limit outgrowth of contaminating Teff cells).

Using this protocol under GMP conditions, researchers can obtain a maximum of 4.5×10⁶/Kg (based on a 250 ml blood draw and an average bodyweight of 80-100 Kg). It has been widely suggested that these numbers may fall short of the 1.5×10⁷/Kg theoretical therapeutic dose for most indications. Therefore, although current expansion methods are sufficient for safety studies, improvements in expansion are required for dose-finding exercises, cryopreservation, and Phase II/III efficacy studies.

The inventors have found that co-culture of Tregs with certain cells, the presence of Treg stimulation agents, resulted in significantly expanded populations Tregs that express high levels of FoxP3 and exhibit increased tissue homing capacity without any compromise in suppression potency, as compared to Tregs cultured under the same conditions but without the certain cells.

Based at least on these findings, the invention provides methods including, but not limited to, methods for enhancing proliferation of Tregs, populations of the proliferated Tregs, compositions comprising the proliferated Tregs, compositions for achieving enhanced expansion of Tregs, methods for immune modulation using the proliferated Tregs, methods for constructing a cell bank using the proliferated Tregs, and drug discovery methods.

Accordingly, one embodiment of the invention includes a method of enhancing proliferation of Tregs in vitro. The method comprises contacting Tregs with certain cells, or conditioned medium from the certain cells, in the presence of one or more Treg stimulation agents. The one or more Treg stimulation agents is present in an amount and for a time effective to stimulate proliferation of the Tregs. The certain cells are present in an amount and for a time effective to enhance proliferation of the Tregs.

In one embodiment, the invention includes a composition comprising Tregs, certain cells, or conditioned medium from the certain cells, and one or more Treg stimulation agents. The one or more Treg stimulation agents is present in an amount effective to stimulate proliferation of the Tregs. The certain cells are present in an amount effective to enhance proliferation of the Tregs.

In one embodiment, the invention includes a composition comprising Tregs, cells (I), or conditioned medium from the cells (I), anti-CD3/anti-CD28-coated beads, IL-2, and rapamycin. The Tregs are CD4+CD14-CD25highCD127low. The cells (I) are non-embryonic, non-germ cells that that have the ability to differentiate into cell types of at least two of endodermal, ectodermal, and mesodermal germ layers and/or express one or more of oct4, telomerase, rex-1 and rox-1. The IL-2 and anti-CD3/anti-CD28-coated beads are present in amounts effective to stimulate proliferation of the Tregs. The cells (I) are present in an amount effective to enhance proliferation of the Tregs.

In one embodiment, the certain cells include, but are not limited to, cells that are not embryonic stem cells and not germ cells, having some characteristics of embryonic stem cells, but being derived from non-embryonic tissue, and providing the effects described in this application. The certain cells may naturally achieve these effects (i.e., not genetically or pharmaceutically modified). However, natural expressors can be genetically or pharmaceutically modified to increase potency.

The certain cells may express pluripotency markers, such as oct4. They may also express markers associated with extended replicative capacity, such as telomerase. Other characteristics of pluripotency can include the ability to differentiate into cell types of more than one germ layer, such as two or three of ectodermal, endodermal, and mesodermal embryonic germ layers. Such certain cells may or may not be immortalized or transformed in culture. The certain cells may be highly expanded without being transformed and also maintain a normal karyotype. For example, in one embodiment, the non-embryonic stem, non-germ cells may have undergone at least 10-40 cell doublings in culture, such as 50, 60, or more, wherein the certain cells are not transformed and have a normal karyotype. The certain cells may differentiate into at least one cell type of each of two of the endodermal, ectodermal, and mesodermal embryonic lineages and may include differentiation into all three. Further, the certain cells may not be tumorigenic, such as not producing teratomas. If the certain cells are transformed or tumorigenic, and it is desirable to use them for infusion, such cells may be disabled so they cannot form tumors in vivo, as by treatment that prevents cell proliferation into tumors. Such treatments are well known in the art.

The certain cells may be prepared by the isolation and culture conditions described herein. In a specific embodiment, they are prepared by culture conditions that are described herein involving lower oxygen concentrations combined with higher serum, such as those used to prepare the certain cells designated “MultiStem®).”

The certain cells include, but are not limited to, the following numbered embodiments:

1. Isolated expanded non-embryonic stem, non-germ cells, the cells having undergone at least 10-40 cell doublings in culture, wherein the cells express oct4, are not transformed or tumorigenic, and have a normal karyotype.

2. The non-embryonic stem, non-germ cells of 1 above that further express one or more of telomerase, rex-1, rox-1, or sox-2.

3. The non-embryonic stem, non-germ cells of 1 above that can differentiate into at least one cell type of at least two of the endodermal, ectodermal, and mesodermal embryonic lineages.

4. The non-embryonic stem, non-germ cells of 3 above that further express one or more of telomerase, rex-1, rox-1, or sox-2.

5. The non-embryonic stem, non-germ cells of 3 above that can differentiate into at least one cell type of each of the endodermal, ectodermal, and mesodermal embryonic lineages.

6. The non-embryonic stem, non-germ cells of 5 above that further express one or more of telomerase, rex-1, rox-1, or sox-2.

7. Isolated expanded non-embryonic stem, non-germ cells that are obtained by culture of non-embryonic, non-germ tissue, the cells having undergone at least 40 cell doublings in culture, wherein the cells are not transformed or tumorigenic and have a normal karyotype.

8. The non-embryonic stem, non-germ cells of 7 above that express one or more of oct4, telomerase, rex-1, rox-1, or sox-2.

9. The non-embryonic stem, non-germ cells of 7 above that can differentiate into at least one cell type of at least two of the endodermal, ectodermal, and mesodermal embryonic lineages.

10. The non-embryonic stem, non-germ cells of 9 above that express one or more of oct4, telomerase, rex-1, rox-1, or sox-2.

11. The non-embryonic stem, non-germ cells of 9 above that can differentiate into at least one cell type of each of the endodermal, ectodermal, and mesodermal embryonic lineages.

12. The non-embryonic stem, non-germ cells of 11 above that express one or more of oct4, telomerase, rex-1, rox-1, or sox-2.

13. Isolated expanded non-embryonic stem, non-germ cells, the cells having undergone at least 10-40 cell doublings in culture, wherein the cells express telomerase, are not transformed or tumorigenic, and have a normal karyotype.

14. The non-embryonic stem, non-germ cells of 13 above that further express one or more of oct4, rex-1, rox-1, or sox-2.

15. The non-embryonic stem, non-germ cells of 13 above that can differentiate into at least one cell type of at least two of the endodermal, ectodermal, and mesodermal embryonic lineages.

16. The non-embryonic stem, non-germ cells of 15 above that further express one or more of oct4, rex-1, rox-1, or sox-2.

17. The non-embryonic stem, non-germ cells of 15 above that can differentiate into at least one cell type of each of the endodermal, ectodermal, and mesodermal embryonic lineages.

18. The non-embryonic stem, non-germ cells of 17 above that further express one or more of oct4, rex-1, rox-1, or sox-2.

19. Isolated expanded non-embryonic stem, non-germ cells that can differentiate into at least one cell type of at least two of the endodermal, ectodermal, and mesodermal embryonic lineages, said cells having undergone at least 10-40 cell doublings in culture, wherein the cells are not transformed or tumorigenic.

20. The non-embryonic stem, non-germ cells of 19 above that express one or more of oct4, telomerase, rex-1, rox-1, or sox-2.

21. The non-embryonic stem, non-germ cells of 19 above that can differentiate into at least one cell type of each of the endodermal, ectodermal, and mesodermal embryonic lineages.

22. The non-embryonic stem, non-germ cells of 21 above that express one or more of oct4, telomerase, rex-1, rox-1, or sox-2.

In one embodiment, the certain cells are derived from bone marrow.

In one embodiment, the certain cells are derived from a human subject.

In one embodiment, the one or more Treg stimulation agents include anti-CD3/anti-CD28 coated beads and/or IL-2. In one example, the one or more Treg stimulation agents include anti-CD3/anti-CD28 coated beads and IL-2.

In one embodiment, the one or more Treg stimulation agents include an antigen-presenting cell.

In one example, the concentration of IL-2 is about 50 IU/ml to about 2000 IU/ml, about 600 IU/ml to about 1200 IU/ml, or at least about 500 IU/ml.

In one example, the ratio of anti-CD3/anti-CD28-coated beads to Tregs is about 1:1.

In one embodiment, the Tregs and the certain cells are contacted with an agent that suppresses T effector cell proliferation. The agent can include, but is not limited to, rapamycin.

In one embodiment, the Tregs are expanded at least about 2-fold to about 1000-fold as compared to control Tregs, at least about 5-fold to about 500-fold as compared to control Tregs, or at least about 6-fold as compared to control Tregs. The control Tregs are cultured under the same conditions as the expanded Tregs but in the absence of certain cells.

In one embodiment, the number of Tregs in the certain cells, prior to proliferation, is about 1,000 cells to about 500,000 cells, or about 100,000 cells to about 300,000 cells, or about 150,000 cells.

In one embodiment, the number of the certain cells in the Tregs, prior to proliferation, is about 1,000 cells to about 500,000 cells, about 100,000 cells to about 300,000 cells, or about 15,000 cells.

In one embodiment, Treg purity can range from about 5% to about 99%, or about 90-99%, or about 99% prior to expansion.

In one embodiment, the Tregs are CD4+CD14-CD25highCD127low.

In one embodiment, the Tregs, after proliferation, express a higher level of α4β7 as compared to control Tregs, which are cultured under the same conditions but in the absence of certain cells. In some embodiments, the Tregs, after proliferation, express a level of α4β7 that is about 1.1-fold to 10-fold higher, about 1.3-fold to 5-fold higher, or about 1.5-fold to 2-fold higher (e.g., 1.7-fold higher) as compared to control Tregs, which are cultured under the same conditions but in the absence of certain cells.

In one embodiment, the Tregs, after proliferation, express a lower level of CCR7 and CD27 as compared to control Tregs, which are cultured under the same conditions but in the absence of certain cells. In some embodiments, the Tregs, after proliferation, express a level of CCR7 that is about 0.1-fold to 10-fold lower, about 0.2-fold to 5-fold lower, or about 0.3-fold to 1-fold lower (e.g., 0.8-fold lower) as compared to control Tregs, which are cultured under the same conditions but in the absence of certain cells. In some embodiments, the Tregs, after proliferation, express a level of CD27 that is about 0.1-fold to 10-fold lower, about 0.2-fold to 5-fold lower, or about 0.3-fold to 1-fold lower (e.g., 0.4-fold lower) as compared to control Tregs, which are cultured under the same conditions but in the absence of certain cells.

In one embodiment, the Tregs, after proliferation, express a higher level of FoxP3 as compared to control Tregs, which are cultured under the same conditions but in the absence of certain cells.

In one embodiment, the level of FoxP3 expressed by the expanded Tregs is about 1.1-fold to 10-fold higher than the level of FoxP3 expressed by control Tregs, about 1.3-fold to 5-fold higher than the level of FoxP3 expressed by control Tregs, or about 1.7-fold to about 2-fold higher than the level of FoxP3 expressed by control Tregs. The control Tregs are cultured under the same conditions as the expanded Tregs but in the absence of certain cells.

In one embodiment, the Tregs, after proliferation, have an increased potency to reduce T effector cell proliferation. In some embodiments, the expanded Tregs increase suppression of T effector cell proliferation by about 0.1-fold to about 10-fold, about 1-fold to about 5-fold, or about 1.5-fold to about 2-fold (e.g., about 1.8-fold) as compared to control Tregs, which are cultured under the same conditions but in the absence of certain cells. One skilled in the art will appreciate that the fold-increase in T effector cell suppression by the expanded Tregs can depend upon the ratio of Tregs to Teffs (in a suppression assay) and the Treg stimulation agent. Where the Treg:Teff ratio is 1:25, for instance, the fold-increase in T effector cell suppression is about 0.1-fold to about 10-fold, about 1-fold to about 5-fold, or about 1.5-fold to about 2-fold (e.g., about 1.8-fold) as compared to control Tregs.

In one embodiment, the ratio of certain cells:Tregs is about 1:10,000 to about 100:1.

In one embodiment, the ratio of certain cells:Tregs is about 1:1,000 to about 5:1.

In one embodiment, the ratio of certain cells:Tregs is about 1:10.

In one embodiment, the Tregs are derived from peripheral blood. In one example, the Tregs that are contacted with the certain cells are in an unfractionated population of PBMCs.

In one embodiment, the Tregs are isolated after proliferation.

In one example, Tregs isolated after proliferation are admixed with a pharmaceutically acceptable carrier.

In one embodiment, the invention includes a method for immune modulation in a subject. The method comprises administering to the subject a therapeutically effective amount of Tregs produced by the methods disclosed herein.

In one embodiment, the immune modulation is effective to treat an aberrant immune response. In one example, the aberrant immune response is an autoimmune disease selected from the group consisting of GVHD, Type 1 diabetes, lupus, multiple sclerosis, asthma, sepsis, and solid organ transplantation.

In one embodiment, the invention includes Tregs produced by the methods disclosed herein.

### BRIEF DESCRIPTION OF THE FIGURES

FIGS. 1A-D—Schematics showing Tregs produced by exposure to MultiStem and standard Tregs were both >99% pure CD3+CD4+ Foxp3+ cells (FIG. 1A), Tregs produced by exposure to MultiStem expanded 6.9-fold greater than standard Tregs (FIG. 1B), Tregs produced by exposure to MultiStem expressed higher levels of FoxP3 (MFI 1500 vs. 825) (FIG. 1C), and Tregs exposure to MultiStem have increased gut homing potential (α4β7) (FIG. 1D).

FIGS. 2A-C—Schematics showing that Tregs produced by the invention suppressed proliferation with equivalent potency to standard Tregs, and that this was true for proliferation driven by anti-CD3/anti-CD28 (synthetic polyclonal Th0 responses) (FIG. 2A), influenza hemagglutinin (Th1 response) (FIG. 2B), and Candida albicans (Th17 response) (FIG. 2C).

### DETAILED DESCRIPTION OF THE INVENTION

It should be understood that this invention is not limited to the particular methodology, protocols, and reagents, etc., described herein and, as such, may vary. The terminology used herein is for the purpose of describing particular embodiments only, and is not intended to limit the scope of the disclosed invention, which is defined solely by the claims.

The section headings are used herein for organizational purposes only and are not to be construed as in any way limiting the subject matter described.

The methods and techniques of the present application are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1993), and Harlow and Lane, Antibodies: A Laboratory Manual, Cold Spring Harbor Laboratory Press, Cold Spring Harbor, N.Y. (1990).

### Definitions

“A” or “an” means herein one or more than one; at least one. Where the plural form is used herein, it generally includes the singular.

“Aberrant immune response” refers to the failure of a subject's immune system to distinguish self from non-self or the failure to respond to foreign antigens. The term also embraces hyperimmune responses to foreign antigens as in the case of allergic disorders. Thus, the response is present in both autoimmune disorders and allergic disorders. Aberrant immune responses include, but are not limited to, tissue injury and inflammation caused by the production of antibodies to an organism's own tissue, impaired production of cytokines and tissue damage caused by cytotoxic or non-cytotoxic mechanisms of action. In some embodiments, aberrant immune responses are inappropriately regulated immune responses that lead to patient symptoms. Typically, autoimmune responses occur when the immune system of a subject recognizes self-antigens as foreign, leading to the production of self-reactive effector immune cells. Self-reactive effector immune cells include cells from a variety of lineages, including, but not limited to, cytotoxic T cells, helper T cells, and B cells. While the precise mechanisms differ, the presence of autoreactive effector immune cells in a patient suffering from an autoimmune disorder may lead to the destruction of tissues and cells of the patient, resulting in pathologic symptoms. Similarly, the presence of cells that undergo a hypersensitive reaction to foreign antigens to which normal individuals respond in a more restrain manner is indicative of hypersensivity (allergy). Examples include, but are not limited to, food allergies, hay fever, and allergic asthma. Numerous assays for determining the presence of such cells in a patient, and therefore the presence of an autoimmune disorder, such as an antigen-specific autoimmune disorder in a patient, or an allergic disorder, are known to those of skill in the art and can be readily employed in the subject methods.

“Antigen presenting cell” refers to a cell that can process a protein antigen, break it into peptides, and present it in conjunction with MHC molecules on the cell surface, where it may interact with appropriate T cell receptors. The term is intended to encompass both professional and non-professional antigen presenting cells. Professional antigen-presenting cells include any antigen presenting cell with highly efficient immunostimulatory capacity. Professional antigen-presenting cells display antigenic peptide fragments in association with the proper class of MHC molecules and also bear costimulatory surface molecules. Different classes of professional antigen-presenting cells include Langherhans' cells, interdigitating cells, follicular dendritic cells, germinal center dendritic cells, B cells and macrophages.

“Autoimmune disease” means a disease or disorder arising from and directed against an individual's own tissues. An autoimmune disease can be selected from the group consisting of: ankylosing spondylitis; Chagas disease; chronic obstructive pulmonary disease; Crohns Disease; idiopathic inflammatory bowel disease; dermatomyositis; diabetes mellitus type 1; endometriosis; Goodpasture's syndrome; graft-versus-host disease (GVHD); Graves' disease; Guillain-Barré syndrome (GBS); Hashimoto's disease; hidradenitis suppurativa; kawasaki disease; IgA nephropathy; idiopathic thrombocytopenic purpura; interstitial cystitis; lupus erythematosus; mixed connective tissue disease; morphea; multiple sclerosis; myasthenia gravis; narcolepsy; neuromyotonia; pemphigus vulgaris; pernicious anemia; psoriasis; psoriatic arthritis; polymyositis; polymyalgia rheumatica; primary biliary cirrhosis; relapsing polychondritis; rheumatoid arthritis; schizophrenia; scleroderma; sclerosing colangitis; Sjögren's syndrome; stiff person syndrome; temporal arteritis; ulcerative colitis; vasculitis Vitiligo; and Wegener's granulomatosis.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created using oligodendrocytes produced by the methods described in this application.

“Co-administer” means to administer in conjunction with one another, together, coordinately, including simultaneous or sequential administration of two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” is a synonym of “comprising” (see above).

“Conditioned medium” refers to a medium that certain cells have been cultured in for a period of time. In one example, conditioned medium is prepared by culturing certain cells (e.g., MAPC) for about 24-72 hours with our without inflammatory cytokines. The supernatant, or conditioned medium, is collected after the culture period. See, for example, Busch, S A et al., J Neurosci. 31(3):944-953 (2011).

“Control cell” generally refers to a cell that is not subjected to or contacted with a test agent or test condition(s), and which serves as a reference cell to determine or evaluate differences in another cell (e.g., a test cell) which has been subject to the test agent or test condition. Typically, a control cell is the same cell type as the test cell (e.g., a T regulatory cell, or Treg, isolated from the same source using the same or similar methods). By way of example, a control Treg is grown or treated via “standard culture conditions” (defined below) or conditions typically used for Treg culture, while a test Treg is subject to one or more variables (e.g., co-culture with certain cells, such as MAPC). In some embodiments, the difference between a test Treg and a control Treg includes, without limitation, differences in phenotype (e.g., FoxP3, CD27, and CCR7 expression), differences in the levels of secreted molecules (e.g., IFNγ and IL-17A), differences in cell activity (e.g., immunosuppressive activity, e.g., of T effector cells), differences in cell proliferation, and differences in homing potential.

“EC cells” were discovered from analysis of a type of cancer called a teratocarcinoma. In 1964, researchers noted that a single cell in teratocarcinomas could be isolated and remain undifferentiated in culture. This type of stem cell became known as an embryonic carcinoma cell (EC cell).

The term “effective amount”, when referring to the amount of certain cells co-cultured with Tregs, refers to an amount of the certain cells sufficient to enhance proliferation of the Tregs.

When referring to methods of immune modulation using the expanded Tregs of the present disclosure, “effective amount” refers to an amount which provides the desired local or systemic effect, e.g., effective for immune modulation, including achieving the specific desired effects described in this application, such as treating an autoimmune disease. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. In the context of immune modulation, the term “effective dose” can mean the same as “effective amount.”

When referring to the amount of a Treg stimulation agent, “effective” refers to an amount of the Treg stimulation agent(s) sufficient to cause proliferation of Tregs.

When referring to the amount of an agent that suppresses T effector cell proliferation, “effective” refers to an amount of the agents sufficient to suppress (reduce) T effector cell proliferation.

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result.

“Effective time”, when referring to the invention, refers to a period of time sufficient to bring about a particular effect, such as, Treg proliferation and enhancement of Treg proliferation.

“Embryonic Stem Cells (ESC)” are well known in the art and have been prepared from many different mammalian species. Embryonic stem cells are stem cells derived from the inner cell mass of an early stage embryo known as a blastocyst. They are able to differentiate into all derivatives of the three primary germ layers: ectoderm, endoderm, and mesoderm. These include each of the more than 220 cell types in the adult body. The ES cells can become any tissue in the body, excluding placenta. Only the morula's cells are totipotent, able to become all tissues and a placenta. Some cells similar to ESCs may be produced by nuclear transfer of a somatic cell nucleus into an enucleated fertilized egg.

“Enhance”, when referring to the invention, means increasing or improving Treg proliferation to a greater degree possible when compared to Treg proliferation under standard culture conditions, which means that those conditions used to proliferate Tregs, but which do not include exposure to the cells (e.g., MAPCs) that enhance proliferation.

“Expression” refers to protein expression or mRNA where the mRNA levels are sufficient to produce protein.

“High” and “low”, when referring to flow cytometry results, refers to the median fluorescent intensity (MFI) of a cell type expressing a particular marker relative to the entire cell population in which cells expressing the particular marker are found. MFI is a measure equal to the geometric mean fluorescence intensity of a log-normal distribution of fluorescence signals. So, “CD25high” means that the MFI of cells expressing CD25 is high relative to the entire cell population (CD4+ cells) in which cells expressing CD25 are found. “CD127low” means that the MFI of cells expressing CD127 is low relative to the entire cell population (CD4+ cells) in which cells expressing CD127 are found.

“Homing”, when used in reference to Tregs expanded by the invention, refers to Tregs that selectively migrate to tissue (e.g., gut tissue) in preference to the lymphatic system (e.g., the thymus).

“Immune response” refers to a patient response to foreign or self antigens. The term includes cell-mediated, humoral, and inflammatory responses.

Use of the term “includes” is not intended to be limiting.

“Increase” or “increasing” means to induce a biological event entirely or to increase the degree of the event.

“Induced pluripotent stem cells (IPSC or IPS cells)” are somatic cells that have been reprogrammed, for example, by introducing exogenous genes that confer on the somatic cell a less differentiated phenotype. These cells can then be induced to differentiate into less differentiated progeny. IPS cells have been derived using modifications of an approach originally discovered in 2006 (Yamanaka, S. et al., Cell Stem Cell, 1:39-49 (2007)). For example, in one instance, to create IPS cells, scientists started with skin cells that were then modified by a standard laboratory technique using retroviruses to insert genes into the cellular DNA. In one instance, the inserted genes were Oct4, Sox2, Lif4, and c-myc, known to act together as natural regulators to keep cells in an embryonic stem cell-like state. These cells have been described in the literature. See, for example, Wernig et al., PNAS, 105:5856-5861 (2008); Jaenisch et al., Cell, 132:567-582 (2008); Hanna et al., Cell, 133:250-264 (2008); and Brambrink et al., Cell Stem Cell, 2:151-159 (2008). These references are incorporated by reference for teaching IPSCs and methods for producing them. It is also possible that such cells can be created by specific culture conditions (exposure to specific agents).

The term “isolated” refers to cells (e.g., Tregs or MAPC) which are not associated with one or more cells or one or more cellular components that are associated with the cells (e.g., Tregs or MAPC) in vivo. An “enriched population” means a relative increase in numbers of a desired cell (e.g., Treg or MAPC) relative to one or more other cell types in vivo or in primary culture.

However, as used herein, the term “isolated” does not indicate the presence of only a particular cell (e.g., Treg or MAPC). Rather, the term “isolated” indicates that the cells (e.g., Tregs or MAPC) are removed from their natural tissue environment and are present at a higher concentration as compared to the normal tissue environment. Accordingly, an “isolated” cell population may further include cell types in addition to particular cells (e.g., Tregs or MAPC) and may include additional tissue components. This also can be expressed in terms of cell doublings, for example. A Treg or MAPC may have undergone 10, 20, 30, 40 or more doublings in vitro or ex vivo so that it is enriched compared to its original numbers in vivo or in its original tissue environment (e.g., bone marrow, peripheral blood, placenta, umbilical cord, umbilical cord blood, adipose tissue, etc.).

“MAPC” is an acronym for “multipotent adult progenitor cell.” It refers to a cell that is not an embryonic stem cell or germ cell but has some characteristics of these. MAPC can be characterized in a number of alternative descriptions, each of which conferred novelty to the cells when they were discovered. They can, therefore, be characterized by one or more of those descriptions. First, they have extended replicative capacity in culture without being transformed (tumorigenic) and with a normal karyotype. Second, they may give rise to cell progeny of more than one germ layer, such as two or all three germ layers (i.e., endoderm, mesoderm and ectoderm) upon differentiation. Third, although they are not embryonic stem cells or germ cells, they may express markers of these primitive cell types so that MAPCs may express one or more of Oct 3/4 (i.e., Oct 3A), rex-1, and rox-1. They may also express one or more of sox-2 and SSEA-4. Fourth, like a stem cell, they may self-renew, that is, have an extended replication capacity without being transformed. This means that these cells express telomerase (i.e., have telomerase activity). Accordingly, the cell type that was designated “MAPC” may be characterized by alternative basic characteristics that describe the cell via some of its novel properties.

The term “adult” in MAPC is non-restrictive. It refers to a non-embryonic somatic cell. MAPCs are karyotypically normal and do not form teratomas in vivo. This acronym was first used in U.S. Pat. No. 7,015,037 to describe a pluripotent cell isolated from bone marrow. However, cells with pluripotential markers and/or differentiation potential have been discovered subsequently and, for purposes of this invention, may be equivalent to those cells first designated “MAPC.” Essential descriptions of the MAPC type of cell are provided in the Summary of the Invention above.

MAPC represents a more primitive progenitor cell population than MSC (Verfaillie, C. M., Trends Cell Biol 12:502-8 (2002); Jahagirdar, B. N., et al., Exp Hematol, 29:543-56 (2001); Reyes, M. and C. M. Verfaillie, Ann N Y Acad Sci, 938:231-233 (2001); Jiang, Y. et al., Exp Hematol, 30896-904 (2002); and Jiang, Y. et al., Nature, 418:41-9. (2002)).

The term “MultiStem®” is the trade name for a cell preparation based on the MAPCs of U.S. Pat. No. 7,015,037, i.e., a non-embryonic stem, non-germ cell as described above. MultiStem® is prepared according to cell culture methods disclosed in this patent application, particularly, lower oxygen and higher serum. MultiStem® is highly expandable, karyotypically normal, and does not form teratomas in vivo. It may differentiate into cell lineages of more than one germ layer and may express one or more of telomerase, oct3/4, rex-1, rox-1, sox-2, and SSEA4.

The terms “peripheral blood mononuclear cell” or “PBMC” refers to a blood cell having a nucleus, such as a lymphocyte or monocyte.

“Pharmaceutically-acceptable carrier” is any pharmaceutically-acceptable medium for the cells used in the present invention. Such a medium may retain isotonicity, cell metabolism, pH, and the like. It is compatible with administration to a subject in vivo, and can be used, therefore, for cell delivery and treatment.

“Primordial embryonic germ cells” (PG or EG cells) can be cultured and stimulated to produce many less differentiated cell types.

The terms “proliferating” and “proliferation” refer to growth and division of cells (e.g., Tregs) in amounts that can be purified after expansion and could be applied in therapeutic applications. In some embodiments, the terms can be used interchangeably with “expand” or “expansion”.

The term “proliferated” with reference to Tregs includes isolated Tregs that have been cultured in vitro with certain cells, in the presence of one or more Treg stimulation agents. In some embodiments, proliferated Tregs express higher levels of FoxP3 as compared to control Tregs. In some embodiments, proliferated Tregs have increased tissue (e.g., gut) homing potential compared to control Tregs. In some embodiments, proliferated Tregs have increased potency to reduce T effector cell proliferation compared to control Tregs.

The term “reduce” as used herein means to prevent as well as decrease. In the context of treatment, to “reduce” is to either prevent or ameliorate one or more clinical symptoms. A clinical symptom is one (or more) that has or will have, if left untreated, a negative impact on the quality of life (health) of the subject. This also applies to the underlying biological effects, such as reduced deletion or enhanced activation of autoreactive CD4⁺ T-helper (Th) lymphocytes, defective immunomodulation by CD4⁺ regulatory (Treg) and CD8⁺ suppressor (Ts) T-lymphocytes, dysregulated signaling (leading to a relative increase in pro-inflammatory cytokines), comparable structure between self-antigens and foreign molecules, and expression of new epitopes on previously hidden or xenobiotic-modified self proteins, the end result of which would be to ameliorate the deleterious clinical symptoms associated with an autoimmune disease. In an in vitro context, to “reduce” is to decrease one or more analytes or biomarkers, which may be assayed and then correlated to a particular outcome or endpoint.

“Self-renewal” of a stem cell refers to the ability to produce replicate daughter stem cells having differentiation potential that is identical to those from which they arose. A similar term used in this context is “proliferation.”

“Standard culture conditions” refers to those conditions, known in the art, which are typically used to culture a given cell type. By way of example, but not by way of limitation, “standard culture conditions” for Tregs include the following: media: RPMI, e.g., RPM1640 (Invitrogen), X-VIVO (Lonza) or equivalent commercial media, supplemented with 5-10% serum (e.g., fetal calf serum (Hyclone) or human serum obtained commercially or from blood of patient/donor) and antibiotics (e.g., penicillin streptomycin and Fungizone); culturing condition: 37° C., 5% CO₂ using, e.g., an incubator capable of controlling oxygen concentration; humidity: >95%; cell density: typically about 5×10⁵ cells/ml; media change: after about 3 days; and Treg stimulation agents: anti-CD3/anti-CD28-coated beads or antigen presenting cells and IL-2 (e.g., 500 IU/ml and above) and rapamycin. Generally, “standard culture conditions” means those conditions under which the Tregs are proliferated but without the cells that enhance proliferation (e.g., MAPC).

“Stem cell” means a cell that can undergo self-renewal (i.e., progeny with the same differentiation potential) and also produce progeny cells that are more restricted in differentiation potential. Within the context of the invention, a stem cell would also encompass a more differentiated cell that has de-differentiated, for example, by nuclear transfer, by fusion with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

Dedifferentiation may also be caused by the administration of certain compounds or exposure to a physical environment in vitro or in vivo that would cause the dedifferentiation. Stem cells also may be derived from abnormal tissue, such as a teratocarcinoma and some other sources such as embryoid bodies (although these can be considered embryonic stem cells in that they are derived from embryonic tissue, although not directly from the inner cell mass). Stem cells may also be produced by introducing genes associated with stem cell function into a non-stem cell, such as an induced pluripotent stem cell.

“Subject” means a vertebrate, such as a mammal, such as a human. Mammals include, but are not limited to, humans, dogs, cats, horses, cows, and pigs.

“Substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially separated from” a population of second substances has a measurably lower content of second substances as compared to the non-separated mixture of first and second substances. In one aspect, at least about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-99% of the second substance is removed from the first substance.

“Suppression,” “inhibition” and “prevention”, when used in the context of an immune response, are used herein in accordance with accepted definitions. For example, “suppression” results when an ongoing immune response is blocked or significantly reduced as compared with the level of immune response that results absent treatment, e.g., by the proliferated Tregs disclosed herein. “Inhibition” refers to blocking the occurrence of an immune response or significantly reducing such response as compared with the level of immune response that results absent treatment, e.g., by the proliferated Tregs disclosed herein. When administered prophylactically, such blockage may be complete so that no targeted immune response occurs, typically referred to as a “prevention” with regard to completely blocking the immune response before onset; or in the present disclosure, the treatment may reduce the effect as compared to the normal untreated state, typically referred to as suppression or inhibition.

“Suppression”, when used in an in vitro context, refers to the ability of Tregs to significantly block or reduce the proliferation of activated T effector cells when the two cell types are co-cultured in vitro.

“Suppression assay”, in the context of the invention, refers to an assay for assessing the potency of Tregs to suppress T effector cell proliferation. For this purpose, Tregs are co-cultured with T effector cells at different ratios in the presence of a stimulus, e.g., a polyclonal stimulus, such as anti-CD3/anti-CD28-coated beads. Tregs alone show a hypoproliferative response (anergy). T effector cells alone show a proliferation response. Co-culture of Tregs with T effector cells results in reduced proliferation of T effector cells. Cell proliferation can be determined by ³H-thymidine incorporation.

The terms “T effector cell” or “Teff” refer to T cells which function to eliminate antigen (e.g., by producing cytokines which modulate the activation of other cells or by cytotoxic activity). The term “effector T cell” includes T helper cells (e.g., Th1 and Th2 cells) and cytotoxic T cells. Th1 cells mediate delayed type hypersensitivity responses and macrophage activation, while Th2 cells provide help to B cells and are critical in the allergic response (Mosmann and Coffman, 1989, Annu. Rev. Immunol. 7, 145-173; Paul and Seder, 1994, Cell 76, 241-251; Arthur and Mason, 1986, J. Exp. Med. 163, 774-786; Paliard et al., 1988, J. Immunol. 141, 849-855; Finkelman et al., 1988, J. Immunol. 141, 2335-2341). Teffs are CD3+CD4+CD25lowCD127+.

The term “therapeutically effective amount” refers to the amount of an agent determined to produce any therapeutic response in a subject. For example, effective anti-inflammatory therapeutic agents may prolong the survivability of the subject, and/or inhibit overt clinical symptoms. Treatments that are therapeutically effective within the meaning of the term as used herein include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms of an autoimmune disease.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

The terms “T regulatory cells” or “Tregs” refer to a subset of CD4⁺ T cells that suppresses the immune response. Specifically, Tregs maintain order in the immune system by enforcing a dominant negative regulation on other immune cells. This includes the secretion of cytokines including IL-10 and TGFβ, which may induce cell-cycle arrest or apoptosis in Teffs and blocking co-stimulation and maturation of dendritic cells. Tregs are also central in maintaining a balance between immune tolerance to self-antigens and antitumor responses (Sakaguchi et al., Cell 133:775-787 (2008)). Tregs are produced mainly in the thymus and require expression of the transcription factor FoxP3 for both development and function (Fontenot et al., Nat. Immunol. 4:330-336 (2003); Hori et al, Science 299: 1057-1061 (2003); Zheng et al., Nat. Immunol. 8:457-462 (2007)). Tregs produce low levels of IL-2, IL-4, IL-5 and IL-12. Tregs also produce TNFα, TGFβ, IFN-γ and IL-10, albeit at lower levels than Teffs. Tregs can be found in the CD4+CD25high population of cells (see, e.g., Waldmann and Cobbold. 2001. Immunity. 14:399). Tregs actively suppress the proliferation and cytokine production of Th1, Th2, or naïve T cells, which have been stimulated in culture with an activating signal (e.g., antigen and antigen presenting cells or with a signal that mimics antigen in the context of MHC, e.g., anti-CD3 antibody plus anti-CD28 antibody).

In one example, Tregs can be recognized in the following way. To recognize Tregs prior to Treg expansion, a population of cells (e.g., PBMCs isolated from peripheral blood) can be isolated (e.g., using flow cytometry) that are CD4+CD14-CD25highCD127low. The CD4+CD14-CD25highCD127low cells can then be assayed for FoxP3 expression and function, such as potency to suppress activated T effector cell proliferation. Assays for determining FoxP3 expression are known (see, e.g., Chauhan, S K et al., J Immunol. 182(1):148-153, 2009). To assess the CD4+CD14-CD25highCD127low cells for potency, a population of T effector cells is purified, activated, and then assayed for proliferation and/or the presence of one or more inflammatory markers. CD4+CD14-CD25highCD127low cells are then titrated into the T effector cells, and the effect(s) on T effector cell proliferation and/or the presence of the inflammatory marker(s) is determined. Other assays for determining potency of putative Tregs include suppression assays as described, for example, by Chauhan et al. (2009) and Collison, L W et al., Methods Mol Biol. 707:21-37 (2011). CD4+CD14-CD25highCD127low cells that are found to express FoxP3 and exhibit an effect on T effector cell proliferation and/or inflammatory marker expression are recognized as Tregs. The same process can be repeated to recognize Tregs proliferated by the invention.

“Treg stimulation agent” refers to any compound, molecule, or cell capable of inducing proliferation of a Treg in vitro. These include, but are not limited to, anti-CD3/anti-CD28 antibodies (e.g., coated on beads), IL-2, and antigen presenting cells. Anti-CD3 antibodies stimulate the T cell receptor, and anti-CD28 antibodies stimulate of the co-stimulatory molecule CD28, both of which are needed to activate T cells and make them proliferate. IL-2 is responsible for the maintenance of Tregs and for the differentiation of CD4+ T cells into defined effector T cell subsets following antigen-mediated activation. One skilled in the art will recognize that the term “Treg stimulation agent” can be synonymous with “T cell stimuli”, “T cell expanders”, or “artificial antigen presenting cells”.

The term “tumorigenic” refers to a cell's ability to form a tumor in vivo and/or in vitro from that cell per se, e.g., the cell itself becomes, or has the potential to become, a tumor.

“Unfractionated PBMCs” refer to a population of PBMCs that is free of non-nucleated cells, such as red blood cells and platelets.

### Stem Cells

One aspect of the present invention includes expanding Tregs in vitro by contacting a population consisting essentially of Tregs with cells for a time and under conditions sufficient to expand the Tregs. Preferably, the Tregs are contacted with stem cells of vertebrate species, such as humans, non-human primates, domestic animals, livestock, and other non-human mammals. These stem cells include, but are not limited to, those cells described below.

Embryonic Stem Cells

The most well studied stem cell is the embryonic stem cell (ESC) as it has unlimited self-renewal and multipotent differentiation potential. These cells are derived from the inner cell mass of the blastocyst or can be derived from the primordial germ cells of a post-implantation embryo (embryonal germ cells or EG cells). ES and EG cells have been derived, first from mouse, and later, from many different animals, and more recently, also from non-human primates and humans. When introduced into mouse blastocysts or blastocysts of other animals, ESCs can contribute to all tissues of the animal. ES and EG cells can be identified by positive staining with antibodies against SSEA1 (mouse) and SSEA4 (human). See, for example, U.S. Pat. Nos. 5,453,357; 5,656,479; 5,670,372; 5,843,780; 5,874,301; 5,914,268; 6,110,739 6,190,910; 6,200,806; 6,432,711; 6,436,701, 6,500,668; 6,703,279; 6,875,607; 7,029,913; 7,112,437; 7,145,057; 7,153,684; and 7,294,508, each of which is incorporated by reference for teaching embryonic stem cells and methods of making and expanding them. Accordingly, ESCs and methods for isolating and expanding them are well-known in the art.

A number of transcription factors and exogenous cytokines have been identified that influence the potency status of embryonic stem cells in vivo. The first transcription factor to be described that is involved in stem cell pluripotency is Oct4. Oct4 belongs to the POU (Pit-Oct-Unc) family of transcription factors and is a DNA binding protein that is able to activate the transcription of genes, containing an octameric sequence called “the octamer motif” within the promoter or enhancer region. Oct4 is expressed at the moment of the cleavage stage of the fertilized zygote until the egg cylinder is formed. The function of Oct3/4 is to repress differentiation inducing genes (i.e., FoxaD3, hCG) and to activate genes promoting pluripotency (FGF4, Utf1, Rex1). Sox2, a member of the high mobility group (HMG) box transcription factors, cooperates with Oct4 to activate transcription of genes expressed in the inner cell mass. It is essential that Oct3/4 expression in embryonic stem cells is maintained between certain levels. Overexpression or downregulation of >50% of Oct4 expression level will alter embryonic stem cell fate, with the formation of primitive endoderm/mesoderm or trophectoderm, respectively. In vivo, Oct4 deficient embryos develop to the blastocyst stage, but the inner cell mass cells are not pluripotent. Instead they differentiate along the extraembryonic trophoblast lineage. Sall4, a mammalian Spalt transcription factor, is an upstream regulator of Oct4, and is therefore important to maintain appropriate levels of Oct4 during early phases of embryology. When Sall4 levels fall below a certain threshold, trophectodermal cells will expand ectopically into the inner cell mass. Another transcription factor required for pluripotency is Nanog, named after a celtic tribe “Tir Nan Og”: the land of the ever young. In vivo, Nanog is expressed from the stage of the compacted morula, is subsequently defined to the inner cell mass and is downregulated by the implantation stage. Downregulation of Nanog may be important to avoid an uncontrolled expansion of pluripotent cells and to allow multilineage differentiation during gastrulation. Nanog null embryos, isolated at day 5.5, consist of a disorganized blastocyst, mainly containing extraembryonic endoderm and no discernable epiblast.

Non-Embryonic Stem Cells

Stem cells have been identified in most tissues. Perhaps the best characterized is the hematopoietic stem cell (HSC). HSCs are mesoderm-derived cells that can be purified using cell surface markers and functional characteristics. They have been isolated from bone marrow, peripheral blood, cord blood, fetal liver, and yolk sac. They initiate hematopoiesis and generate multiple hematopoietic lineages. When transplanted into lethally-irradiated animals, they can repopulate the erythroid neutrophil-macrophage, megakaryocyte, and lymphoid hematopoietic cell pool. They can also be induced to undergo some self-renewal cell division. See, for example, U.S. Pat. Nos. 5,635,387; 5,460,964; 5,677,136; 5,750,397; 5,681,599; and 5,716,827. U.S. Pat. No. 5,192,553 reports methods for isolating human neonatal or fetal hematopoietic stem or progenitor cells. U.S. Pat. No. 5,716,827 reports human hematopoietic cells that are Thy-1⁺ progenitors, and appropriate growth media to regenerate them in vitro. U.S. Pat. No. 5,635,387 reports a method and device for culturing human hematopoietic cells and their precursors. U.S. Pat. No. 6,015,554 describes a method of reconstituting human lymphoid and dendritic cells. Accordingly, HSCs and methods for isolating and expanding them are well-known in the art.

Another stem cell that is well-known in the art is the neural stem cell (NSC). These cells can proliferate in vivo and continuously regenerate at least some neuronal cells. When cultured ex vivo, neural stem cells can be induced to proliferate as well as differentiate into different types of neurons and glial cells. When transplanted into the brain, neural stem cells can engraft and generate neural and glial cells. See, for example, Gage F. H., Science, 287:1433-1438 (2000), Svendsen S. N. et al, Brain Pathology, 9:499-513 (1999), and Okabe S. et al., Mech Development, 59:89-102 (1996). U.S. Pat. No. 5,851,832 reports multipotent neural stem cells obtained from brain tissue. U.S. Pat. No. 5,766,948 reports producing neuroblasts from newborn cerebral hemispheres. U.S. Pat. Nos. 5,564,183 and 5,849,553 report the use of mammalian neural crest stem cells. U.S. Pat. No. 6,040,180 reports in vitro generation of differentiated neurons from cultures of mammalian multipotential CNS stem cells. WO 98/50526 and WO 99/01159 report generation and isolation of neuroepithelial stem cells, oligodendrocyte-astrocyte precursors, and lineage-restricted neuronal precursors. U.S. Pat. No. 5,968,829 reports neural stem cells obtained from embryonic forebrain. Accordingly, neural stem cells and methods for making and expanding them are well-known in the art.

Another stem cell that has been studied extensively in the art is the mesenchymal stem cell (MSC). MSCs are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

Another example of an adult stem cell is adipose-derived adult stem cells (ADSCs) which have been isolated from fat, typically by liposuction followed by release of the ADSCs using collagenase. ADSCs are similar in many ways to MSCs derived from bone marrow, except that it is possible to isolate many more cells from fat. These cells have been reported to differentiate into bone, fat, muscle, cartilage, and neurons. A method of isolation has been described in U.S. 2005/0153442.

Other stem cells that are known in the art include gastrointestinal stem cells, epidermal stem cells, and hepatic stem cells, which have also been termed “oval cells” (Potten, C., et al., Trans R Soc Lond B Biol Sci, 353:821-830 (1998), Watt, F., Trans R Soc Lund B Biol Sci, 353:831 (1997); Alison et al., Hepatology, 29:678-683 (1998).

Other non-embryonic cells reported to be capable of differentiating into cell types of more than one embryonic germ layer include, but are not limited to, cells from umbilical cord blood (see U.S. Publication No. 2002/0164794), placenta (see U.S. Publication No. 2003/0181269, umbilical cord matrix (Mitchell, K. E. et al., Stem Cells, 21:50-60 (2003)), small embryonic-like stem cells (Kucia, M. et al., J Physiol Pharmacol, 57 Suppl 5:5-18 (2006)), amniotic fluid stem cells (Atala, A., J Tissue Regen Med, 1:83-96 (2007)), skin-derived precursors (Toma et al., Nat Cell Biol, 3:778-784 (2001)), and bone marrow (see U.S. Publication Nos. 2003/0059414 and 2006/0147246), each of which is incorporated by reference for teaching these cells.

Strategies of Reprogramming Somatic Cells

Several different strategies such as nuclear transplantation, cellular fusion, and culture induced reprogramming have been employed to induce the conversion of differentiated cells into an embryonic state. Nuclear transfer involves the injection of a somatic nucleus into an enucleated oocyte, which, upon transfer into a surrogate mother, can give rise to a clone (“reproductive cloning”), or, upon explantation in culture, can give rise to genetically matched embryonic stem (ES) cells (“somatic cell nuclear transfer,” SCNT). Cell fusion of somatic cells with ES cells results in the generation of hybrids that show all features of pluripotent ES cells. Explantation of somatic cells in culture selects for immortal cell lines that may be pluripotent or multipotent. At present, spermatogonial stem cells are the only source of pluripotent cells that can be derived from postnatal animals. Transduction of somatic cells with defined factors can initiate reprogramming to a pluripotent state. These experimental approaches have been extensively reviewed (Hochedlinger and Jaenisch, Nature, 441:1061-1067 (2006) and Yamanaka, S., Cell Stem Cell, 1:39-49 (2007)).

Nuclear Transfer

Nuclear transplantation (NT), also referred to as somatic cell nuclear transfer (SCNT), denotes the introduction of a nucleus from a donor somatic cell into an enucleated ogocyte to generate a cloned animal such as Dolly the sheep (Wilmut et al., Nature, 385:810-813 (1997). The generation of live animals by NT demonstrated that the epigenetic state of somatic cells, including that of terminally differentiated cells, while stable, is not irreversible fixed but can be reprogrammed to an embryonic state that is capable of directing development of a new organism. In addition to providing an exciting experimental approach for elucidating the basic epigenetic mechanisms involved in embryonic development and disease, nuclear cloning technology is of potential interest for patient-specific transplantation medicine.

Fusion of Somatic Cells and Embryonic Stem Cells

Epigenetic reprogramming of somatic nuclei to an undifferentiated state has been demonstrated in murine hybrids produced by fusion of embryonic cells with somatic cells. Hybrids between various somatic cells and embryonic carcinoma cells (Solter, D., Nat Rev Genet, 7:319-327 (2006), embryonic germ (EG), or ES cells (Zwaka and Thomson, Development, 132:227-233 (2005)) share many features with the parental embryonic cells, indicating that the pluripotent phenotype is dominant in such fusion products. As with mouse (Tada et al., Curr Biol, 11:1553-1558 (2001)), human ES cells have the potential to reprogram somatic nuclei after fusion (Cowan et al., Science, 309:1369-1373(2005)); Yu et al., Science, 318:1917-1920 (2006)). Activation of silent pluripotency markers such as Oct4 or reactivation of the inactive somatic X chromosome provided molecular evidence for reprogramming of the somatic genome in the hybrid cells. It has been suggested that DNA replication is essential for the activation of pluripotency markers, which is first observed 2 days after fusion (Do and Scholer, Stem Cells, 22:941-949 (2004)), and that forced overexpression of Nanog in ES cells promotes pluripotency when fused with neural stem cells (Silva et al., Nature, 441:997-1001 (2006)).

Culture-Induced Reprogramming

Pluripotent cells have been derived from embryonic sources such as blastomeres and the inner cell mass (ICM) of the blastocyst (ES cells), the epiblast (EpiSC cells), primordial germ cells (EG cells), and postnatal spermatogonial stem cells (“maGSCsm” “ES-like” cells). The following pluripotent cells, along with their donor cell/tissue is as follows: parthogenetic ES cells are derived from murine oocytes (Narasimha et al., Curr Biol, 7:881-884 (1997)); embryonic stem cells have been derived from blastomeres (Wakayama et al., Stem Cells, 25:986-993 (2007)); inner cell mass cells (source not applicable) (Eggan et al., Nature, 428:44-49 (2004)); embryonic germ and embryonal carcinoma cells have been derived from primordial germ cells (Matsui et al., Cell, 70:841-847 (1992)); GMCS, maSSC, and MASC have been derived from spermatogonial stem cells (Guan et al., Nature, 440:1199-1203 (2006); Kanatsu-Shinohara et al., Cell, 119:1001-1012 (2004); and Seandel et al., Nature, 449:346-350 (2007)); EpiSC cells are derived from epiblasts (Brons et al., Nature, 448:191-195 (2007); Tesar et al., Nature, 448:196-199(2007)); parthogenetic ES cells have been derived from human oocytes (Cibelli et al., Science, 295L819 (2002); Revazova et al., Cloning Stem Cells, 9:432-449 (2007)); human ES cells have been derived from human blastocysts (Thomson et al., Science, 282:1145-1147 (1998)); MAPC have been derived from bone marrow (Jiang et al., Nature, 418:41-49 (2002); Phinney and Prockop, Stem Cells, 25:2896-2902 (2007)); cord blood cells (derived from cord blood) (van de Ven et al., Exp Hematol, 35:1753-1765 (2007)); neurosphere derived cells derived from neural cell (Clarke et al., Science, 288:1660-1663 (2000)). Donor cells from the germ cell lineage such as PGCs or spermatogonial stem cells are known to be unipotent in vivo, but it has been shown that pluripotent ES-like cells (Kanatsu-Shinohara et al., Cell, 119:1001-1012 (2004) or maGSCs (Guan et al., Nature, 440:1199-1203 (2006), can be isolated after prolonged in vitro culture. While most of these pluripotent cell types were capable of in vitro differentiation and teratoma formation, only ES, EG, EC, and the spermatogonial stem cell-derived maGCSs or ES-like cells were pluripotent by more stringent criteria, as they were able to form postnatal chimeras and contribute to the germline. Recently, multipotent adult spermatogonial stem cells (MASCs) were derived from testicular spermatogonial stem cells of adult mice, and these cells had an expression profile different from that of ES cells (Seandel et al., Nature, 449:346-350 (2007)) but similar to EpiSC cells, which were derived from the epiblast of postimplantation mouse embryos (Brons et al., Nature, 448:191-195 (2007); Tesar et al., Nature, 448:196-199 (2007)).

Reprogramming by Defined Transcription Factors

Takahashi and Yamanaka have reported reprogramming somatic cells back to an ES-like state (Takahashi and Yamanaka, Cell, 126:663-676 (2006)). They successfully reprogrammed mouse embryonic fibroblasts (MEFs) and adult fibroblasts to pluripotent ES-like cells after viral-mediated transduction of the four transcription factors Oct4, Sox2, c-myc, and Klf4 followed by selection for activation of the Oct4 target gene Fbx15. Cells that had activated Fbx15 were coined iPS (induced pluripotent stem) cells and were shown to be pluripotent by their ability to form teratomas, although they were unable to generate live chimeras. This pluripotent state was dependent on the continuous viral expression of the transduced Oct4 and Sox2 genes, whereas the endogenous Oct4 and Nanog genes were either not expressed or were expressed at a lower level than in ES cells, and their respective promoters were found to be largely methylated. This is consistent with the conclusion that the Fbx15-iPS cells did not correspond to ES cells but may have represented an incomplete state of reprogramming. While genetic experiments had established that Oct4 and Sox2 are essential for pluripotency (Chambers and Smith, Oncogene, 23:7150-7160 (2004); Ivanona et al., Nature, 442:5330538 (2006); Masui et al., Nat Cell Biol, 9:625-635 (2007)), the role of the two oncogenes c-myc and Klf4 in reprogramming is less clear. Some of these oncogenes may, in fact, be dispensable for reprogramming, as both mouse and human iPS cells have been obtained in the absence of c-myc transduction, although with low efficacy (Nakagawa et al., Nat Biotechnol, 26:191-106 (2008); Werning et al., Nature, 448:318-324 (2008); Yu et al., Science, 318: 1917-1920 (2007)).

### MAPC

Human MAPCs are described in U.S. Pat. No. 7,015,037. MAPCs have been identified in other mammals. Murine MAPCs, for example, are also described in U.S. Pat. No. 7,015,037. Rat MAPCs are also described in U.S. Pat. No. 7,838,289.

These references are incorporated by reference for describing MAPCs first isolated by Catherine Verfaillie.

### Isolation and Growth of MAPCs

Methods of MAPC isolation are known in the art. See, for example, U.S. Pat. No. 7,015,037, and these methods, along with the characterization (phenotype) of MAPCs, are incorporated herein by reference. MAPCs can be isolated from multiple sources, including, but not limited to, bone marrow, placenta, umbilical cord and cord blood, muscle, brain, liver, spinal cord, blood or skin. It is, therefore, possible to obtain bone marrow aspirates, brain or liver biopsies, and other organs, and isolate the cells using positive or negative selection techniques available to those of skill in the art, relying upon the genes that are expressed (or not expressed) in these cells (e.g., by functional or morphological assays such as those disclosed in the above-referenced applications, which have been incorporated herein by reference).

In some embodiments the purity of MAPCs is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the MAPCs have undergone, for example, 1-5, 5-10, 10-20, or more cell doublings.

MAPCs have also been obtained by modified methods described in Breyer et al., Experimental Hematology, 34:1596-1601 (2006) and Subramanian et al., Cellular Programming and Reprogramming: Methods and Protocols; S. Ding (ed.), Methods in Molecular Biology, 636:55-78 (2010), incorporated by reference for these methods.

MAPCs from Human Bone Marrow as Described in U.S. Pat. No. 7,015,037

MAPCs do not express the common leukocyte antigen CD45 or erythroblast specific glycophorin-A (Gly-A). The mixed population of cells was subjected to a Ficoll Hypaque separation. The cells were then subjected to negative selection using anti-CD45 and anti-Gly-A antibodies, depleting the population of CD45⁺ and Gly-A⁺ cells, and the remaining approximately 0.1% of marrow mononuclear cells were then recovered. Cells could also be plated in fibronectin-coated wells and cultured as described below for 2-4 weeks to deplete the cells of CD45⁺ and Gly-A⁺ cells. In cultures of adherent bone marrow cells, many adherent stromal cells undergo replicative senescence around cell doubling 30 and a more homogenous population of cells continues to expand and maintains long telomeres.

Alternatively, positive selection could be used to isolate cells via a combination of cell-specific markers. Both positive and negative selection techniques are available to those of skill in the art, and numerous monoclonal and polyclonal antibodies suitable for negative selection purposes are also available in the art (see, for example, Leukocyte Typing V, Schlossman, et al., Eds. (1995) Oxford University Press) and are commercially available from a number of sources.

Techniques for mammalian cell separation from a mixture of cell populations have also been described by Schwartz, et al., in U.S. Pat. No. 5,759,793 (magnetic separation), Basch et al., 1983 (immunoaffinity chromatography), and Wysocki and Sato, 1978 (fluorescence-activated cell sorting).

Cells may be cultured in low-serum or serum-free culture medium. Serum-free medium used to culture MAPCs is described in U.S. Pat. No. 7,015,037. Commonly-used growth factors include but are not limited to platelet-derived growth factor and epidermal growth factor. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference for teaching growing cells in serum-free medium.

### Additional Culture Methods

In additional experiments the density at which MAPCs are cultured can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Also, effective atmospheric oxygen concentrations of less than about 10%, including about 1-5% and, especially, 3-5%, can be used at any time during the isolation, growth and differentiation of MAPCs in culture.

Cells may be cultured under various serum concentrations, e.g., about 2-20%. Fetal bovine serum may be used. Higher serum may be used in combination with lower oxygen tensions, for example, about 15-20%. Cells need not be selected prior to adherence to culture dishes. For example, after a Ficoll gradient, cells can be directly plated, e.g., 250,000-500,000/cm². Adherent colonies can be picked, possibly pooled, and expanded.

In one embodiment, high serum (around 15-20%) and low oxygen (around 3-5%) conditions can be used for the cell culture. Specifically, adherent cells from colonies can be plated and passaged at densities of about 1700-2300 cells/cm² in 18% serum and 3% oxygen (with PDGF and EGF).

In an embodiment specific for MAPCs, supplements are cellular factors or components that allow MAPCs to retain the ability to differentiate into cell types of more than one embryonic lineage, such as all three lineages. This may be indicated by the expression of specific markers of the undifferentiated state, such as Oct 3/4 (Oct 3A) and/or markers of high expansion capacity, such as telomerase.

### Proliferated Tregs

In one embodiment, the present disclosure is directed to a method of enhancing proliferation of Tregs. In some embodiments, a subject's own Tregs are collected, enriched, and subjected to proliferation protocols according to the methods disclosed herein. In some embodiments, the proliferated Tregs are then administered to the subject, e.g., to treat or ameliorate an aberrant immune response, such as an autoimmune or allergic disorder.

Collection and Enrichment of Tregs

Tregs may be obtained from a variety of sources including, but not limited to, mammals typically used in experimental settings, such as rodents (e.g., mice, rats), rabbits, goats, ferrets, monkeys and apes, common domestic animals, such as cattle, horses, sheep, hogs, dogs, cats, and other mammals, such as those kept in zoos or as pets, etc. In some embodiments, Tregs are isolated from a sample of a subject's peripheral blood, such as from a heterogeneous population of PBMCs. In some embodiments, the subject is a human and the Tregs are human cells. Methods for collecting blood samples and isolating cells are known in the art.

In some embodiments, prior to proliferation, Tregs are substantially separated from the other cells in the blood sample to form a purified Treg population. Methods for isolating and purifying Tregs are known in the art. By way of example, but not by way of limitation, methods may be based on using monoclonal antibodies against cell surface proteins which are predominantly expressed on Tregs. For example, using fluorochrome-conjugated antibodies, Tregs can be labeled and isolated, e.g., by magnetic cell sorting, flow cytometry, etc., (see, e.g., Kawano Y, et al. 2011. Blood 118:5021-5030).

In some embodiments, Tregs are isolated and enriched for one or more of CD3+, CD4+, CD14-, CD25high, CD35+, CD127low cells. In one example, about 70-75%, about 75-80%, about 80-85%, about 85-90%, about 90-95%, or about 95-99% of the isolated and enriched Tregs are CD4+CD14-CD25highCD127low.

By way of example, but not by way of limitation, in some embodiments, human PBMCs are separated from peripheral blood by density centrifugation using Ficoll. The PBMCs can be used, as such, in the Treg expansion protocol described in this application. In one specific embodiment, the procedure is as follows. Whole blood (e.g., about 17 ml) is pipetted in an appropriate number of 50 ml conical tubes. The whole blood is diluted 1:1 or 1:2 in a sterile isotonic solution (e.g., PBS, saline, or serum-free culture media). 10 ml of Ficoll Histopaque is underlayed in each tube. The tubes are then centrifuged at 1600 rpm for 20-40 minutes at 4° C. with no brake. After centrifugation, each tube will have four bands. From bottom to top they are red blood cells, Ficoll, PBMCs, and serum. Centrifuging with the brake activated will cause the PBMC layer to spread throughout the Ficoll band. Shortly after the centrifuge has stopped, the PBMC layer from each tube is removed with a pipette and saved. Other blood layers may be disposed of. Allowing the samples to sit for an extended period of time after centrifugation will also lead to dispersal of the PBMC layer through the Ficoll. Care should be taken to not aspirate any of the red blood cell layer into the pipette. The PBMCs are then washed with an isotonic solution (e.g., PBS, saline, or serum-free culture media). Samples are then centrifuged at 1600 rpm for 5 minutes at 4° C. Next, the supernatant is removed from each tube and the cells are re-suspended in media. The cell count is measured using a hemocytometer. Residual red blood cells or dead cells are not counted. If needed, residual red blood cells may be lysed.

After isolation, the PBMCs are contacted with antibodies (e.g., anti-CD4, anti-CD25 and anti-CD127 antibodies) and cells (e.g., CD4+CD14-CD25highCD127low) are isolated as Tregs by, e.g., FACS Aria II Cell Sorter. In some embodiments, about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-99% of the isolated and enriched Tregs are CD4+CD14-CD25highCD127low.

In some embodiments, Tregs may be selected against dead cells by employing dyes associated with dead cells (e.g., propidium iodide, ethidium monoazaide). Any technique may be employed which is not unduly detrimental to the viability of the selected cells.

In some embodiments, cell sorting is used. In some embodiments, Tregs may be collected in any appropriate medium that maintains the viability of the cells, usually having a cushion of serum at the bottom of the collection tube. Various media are commercially available and may be used according to the nature of the cells, including Dulbecco's Modified Eagle Medium (“dMEM”), Hank's Basic salt Solution (“HBSS”), Dulbecco's phosphate buffered saline (“dPBS”), RPMI, Iscove's medium, etc., frequently supplemented with fetal calf serum.

Proliferation of Tregs

The culture conditions disclosed herein are used to enhance proliferation of Tregs, which are useful as therapeutic agents. The culture conditions include one or more of the following: (a) contacting a population of Tregs with cells (e.g., MAPCs), in the presence of one or more Treg stimulation agents, wherein the one or more Treg stimulation agents is present in an amount effective to stimulate proliferation of the Tregs, and wherein the cells are present in an amount effective to enhance proliferation of the Tregs; and (b) contacting the Tregs and cells with an agent that suppresses Teff proliferation. In one embodiment, the Tregs that are exposed to the cells (e.g., MAPC) can be an enriched or purified population (e.g., CD4+CD14-CD25highCD127low cells). In another embodiment, the Tregs that are exposed to the cells (e.g., MAPC) can comprise part of an unfractionated population of PBMCs. Such proliferated Tregs exhibit higher levels of FoxP3 expression, increased tissue (e.g., gut) homing potential, and increased potency to reduce Teff proliferation as compared to control Tregs (e.g., Tregs that were proliferated under standard culture conditions but in the absence of cells, e.g., MAPC).

In one embodiment, Tregs are co-cultured with cells (e.g., MAPC), in the presence of one or more Treg stimulation agents, under standard temperature and humidity conditions and in standard growth medium (e.g., RPMI). Initially, cells (e.g., MAPC) and Tregs can each be seeded at a desired density (or absolute cell number) and at a desired ratio. In one example, cells (e.g., MAPC) are seeded (in the Tregs) at an absolute cell number of about 1,000-50,000 cells, about 50,000-100,000 cells, about 100,000-150,000 cells, about 150,000-200,000 cells, about 200,000-250,000 cells, about 250,000-300,000 cells, about 300,000-350,000 cells, about 350,000-400,000 cells, about 400,000-450,000 cells, or about 450,000-500,000 or more cells. In one example, cells (e.g., MAPC) are seeded at an absolute cell number of about 15,000 cells.

In some embodiments, Tregs are seeded (in the cells, e.g., MAPC) at an absolute cell number of about 1,000-50,000 cells, about 50,000-100,000 cells, about 100,000-150,000 cells, about 150,000-200,000 cells, about 200,000-250,000 cells, about 250,000-300,000 cells, about 300,000-350,000 cells, about 350,000-400,000 cells, about 400,000-450,000 cells, or about 450,000-500,000 cells. In one example, Tregs are seeded at an absolute cell number of about 150,000 cells.

In some embodiments, the ratio of cells (e.g., MAPC) to Tregs in initial co-culture can be about 1:10,000 to about 100:1, about 1:9,000 to about 90:1, about 1:8,000 to about 80:1, about 1:7,000 to about 70:1, about 1:6,000 to about 60:1, about 1:5,000 to about 50:1, about 1:4,000 to about 40:1, about 1:3,000 to about 30:1, about 1:2,000 to about 20:1, or about 1:1,000 to about 5:1. In one example, the ratio of cells (e.g., MAPC) to Tregs in initial co-culture is about 1:10.

In some embodiments, the purity of the cells (e.g., MAPC) is about 100% (substantially homogeneous). In some embodiments, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells (e.g., MAPC) have undergone, for example, 1-5, 5-10, 10-20, or more cell doublings.

The population of Tregs and cells (e.g., MAPC) can be co-cultured, in the presence of one or more Treg stimulation agents, for a desired period of time before harvesting the proliferated Tregs. In one embodiment, Tregs and cells (e.g., MAPC) can be co-cultured, in the presence of one or more Treg stimulation agents, for a first period of time (e.g., about 10 days), the proliferated Tregs collected, and then re-plated and co-cultured with cells (e.g., MAPC), in the presence of one or more Treg stimulation agents, for one, two, three, or more periods of time that are equal to or different than the first period of time. The cells (e.g., MAPC) can be added into culture at the beginning of each time period or added back every 2-3 days. The ratio of cells (e.g., MAPC) to Tregs for each time period can be the same or different when compared to other time periods. In one example, Tregs and cells (e.g., MAPC) can be co-cultured for a first period of time (10 days) in the presence of one or more Treg stimulation agents, the proliferated Tregs collected and then re-plated and co-cultured with cells (e.g., MAPC), in the presence of one or more Treg stimulation agents, for a second period of time (10 days), the proliferated Tregs collected and then re-plated and co-cultured with cells (e.g., MAPC), in the presence of one or more Treg stimulation agents, for a third period of time (10 days), whereafter the proliferated Tregs are finally collected. One skilled in the art can determine the total amount of time and sub-periods of time that are optimal by assaying for Treg expansion.

In some embodiments, co-culture of Tregs with cells (e.g., MAPC), in the presence of one or more Treg stimulation agents, can result in proliferation of the Tregs about 2-fold to about 100-fold, about 100-fold to about 200-fold, about 200-fold to about 300-fold, about 300-fold to about 400-fold, about 400-fold to about 500-fold, about 500-fold to about 600-fold, about 600-fold to about 700-fold, about 700-fold to about 800-fold, about 800-fold to about 900-fold, or about 900-fold to about 1000-fold as compared to proliferation of control Tregs cells (e.g., Tregs that were not proliferated according to the methods disclosed herein).

In some embodiments, the Tregs are co-cultured with cells (e.g., MAPC) in the presence of one or more Treg stimulation agents capable of inducing Treg proliferation.

In one embodiment, the Treg stimulation agent is a T-cell receptor (TCR)/CD3 activator, such as an anti-CD3 antibody (e.g., a polyclonal or monoclonal antibody). A number of anti-CD3 monoclonal antibodies are commercially available, e.g., OKT3 and G19-4 monoclonal antibodies prepared from hybridoma cells obtained from the American Type Culture Collection. In another example, the Treg stimulation agent is an anti-CD28 antibody (e.g., a polyclonal antibody or a monoclonal antibody).

In some embodiments, the Treg stimulation agent(s) (e.g., anti-CD3 and anti-CD28 antibodies) may be in soluble form or immobilized on a solid support, such as a bead or tissue culture dish. Anti-CD3/anti-CD28 beads are a commercial preparation (e.g., available from Dynal, ThermoFisher) of magnetic beads coupled to anti-CD3 and anti-CD28 antibodies, which are used to provide signal 1 (anti-CD3; stimulation of the T cell receptor) and signal 2 (stimulation of the co-stimulatory molecule CD28) needed to activate T cells and make them proliferate. In vivo, these signals are provided by antigen presenting cells, so such beads are often called “artificial antigen presenting cells”. Microbeads conjugated with anti-CD3 and anti-CD28 antibodies are commercially available and may be used according to manufacturer's instruction. In some embodiments, the bead-total cell ratio is about 10:1, about 9:1, about 8:1, about 7:1, about 6:1, about 5:1, about 4:1, about 3:1, about 2:1, or about 1:1. In one example, the ratio of anti-CD3/anti-CD28-coated beads to the number of total cells (Tregs and MAPC) initially seeded is 1:1. In some embodiments, the two stimulating agents are coupled to the same solid phase surface, such as a bead, or the bottom of a culture dish or well. The solid phase surface can be plastic, glass, or any other suitable material. In some embodiments, paramagnetic beads are used, and are typically in the 1-20 micron range.

In some embodiments, the Treg stimulation agent is IL-2, IL-7, IL-10, TGF-β, or glucocorticoid-induced TNF-α receptor-related protein ligand (GITR-L). IL-2, for example, is a human cytokine that is critical for normal immune function in vivo and, in vitro, is necessary for T cell growth and Treg survival/expansion. In some embodiments, the concentration of IL-2 used in the invention is about 50-250 IU/ml, about 250-450 IU/ml, about 450-650 IU/ml, about 650-850 IU/ml, about 1050-1250 IU/ml, about 1250-1450 IU/ml, about 1450-1650 IU/ml, about 1650-1850 IU/ml, or about 1850-2000 IU/ml. In one example, the concentration of IL-2 used in the invention is at least about 500 IU/ml. IL-2 is commercially available, for example, as Proleukin (aldesleukin).

In some embodiments, the Treg stimulation agent is an antigen-presenting cell, such as a B-cell or a dendritic cell. See, for example, Yamazaki, S. et al., J Exp Med. 198(2):235-47 (2003), and Yamazaki, S. et al., Immunol Review 212:314-29 (2006).

In one example, Tregs are co-cultured with cells (e.g., MAPC) in the presence of anti-CD3/anti-CD28-coated beads and IL-2. The ratio of anti-CD3/anti-CD28-coated beads to number of cells in culture can be about 1:1, and the concentration of IL-2 can be at least about 500 IU/ml.

In some embodiments, Tregs are co-cultured with cells (e.g., MAPC), in the presence of one or more Treg stimulation agents, for at least about 3-5 days, about 5-8 days, about 8-11 days, about 11-14 days, about 14-17 days, about 17-20 days, about 20-23 days, about 23-26 days, about 26-29 days, about 29-31 days, or about 31-34 or more days.

Additionally or alternatively, in some embodiments, a population of Tregs and cells (e.g., MAPC), in the presence of a Treg stimulation agent, is cultured in the presence of one or more agents that suppress Teff proliferation. Examples of agents that suppresses Teff proliferation include, but are not limited to, naturally-occurring soluble molecules (e.g., IL-10, TGFβ, prostaglandin E2, indoleamine 2,3-dioxygenase), cell surface proteins (e.g., CTLA-4, CD39, CD73, Tim-3), naturally-occurring and in vitro-generated regulatory cells (e.g., regulatory B cells, myeloid-derived suppressor cells, stromal cells, M2 macrophages, mesenchymal stromal cells, tolerogenic dendritic cells, CD8 suppressor T cells, N2 neutrophils), and pharmacological agents (e.g., rapamycin, cyclosporin, mycophenolate mofetil, tacrolimus). In one example, a population of Tregs and cells (e.g., MAPC), in the presence of a Treg stimulation agent, is cultured and expanded in the presence of rapaymycin at a concentration of about 50-75 ng/ml, about 75-100 ng/ml, about 100-125 ng/ml, about 125-150 ng/ml, about 150-175 ng/ml, about 175-200 ng/ml, about 200-225 ng/ml, or about 225-250 ng/ml.

In some embodiments, Tregs are co-cultured with cells (e.g., MAPC), in the presence of a Treg stimulation agent and an agent that suppresses Teffs, for at least about 3-5 days, about 5-8 days, about 8-11 days, about 11-14 days, about 14-17 days, about 17-20 days, about 20-23 days, about 23-26 days, about 26-29 days, about 29-31 days, or about 31-34 or more days.

In another embodiment, the Tregs that are exposed to the cells (e.g., MAPC) can be found in an unfractionated population of PBMCs. In this embodiment, PBMCs are isolated from peripheral blood as described above. The isolated PBMCs are then placed in a culture medium (e.g., RPMI-1640 with 10% FBS) and contacted with cells (e.g., MAPC). The ratio of PBMCs to the cells in the culture medium can about 1:1, about 1:50, about 1:100, about 1:200; about 1:300, or about 1:400. In one example, the ratio PBMCs to the cells is 1:128. The PBMCs and the cells are then cultured for a period of time of about 1-2 days, about 2-3 days, about 3-4 days, about 4-5 days, about 5-6 days, or about 7-8 or more days. In one example, the PBMCs and the cells are cultured for 6 days. After culturing for the period of time, Tregs are isolated using, for example, flow cytometry as described above. In this embodiment, an exogenous Treg stimulation agent (e.g., IL-2 and/or anti-CD3/anti-CD28-coated beads) may not need to be applied.

### Pharmaceutical Formulations

Expanded Tregs produced by the present invention can be formulated as a pharmaceutical composition.

U.S. Pat. No. 7,015,037 is incorporated by reference for teaching pharmaceutical formulations. In certain embodiments, the expanded Tregs are present within a composition adapted for and suitable for delivery, i.e., physiologically compatible.

In some embodiments the purity of the expanded Tregs for administration to a subject is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the Tregs have undergone, for example, 1-5, 5-10, 10-20, or more cell doublings.

The choice of formulation for administering the expanded Tregs for a given application will depend on a variety of factors. Prominent among these will be the species of subject, the nature of the autoimmune disease or aberrant immune response being treated, its state and distribution in the subject, the nature of other therapies and agents that are being administered, the optimum route for administration, survivability via the route, the dosing regimen, and other factors that will be apparent to those skilled in the art. For instance, the choice of suitable carriers and other additives will depend on the exact route of administration and the nature of the particular dosage form.

Final formulations of the aqueous suspension of cells/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant.

In some embodiments, the expanded Tregs are formulated in a unit dosage injectable form, such as a solution, suspension, or emulsion. Pharmaceutical formulations suitable for injection of the expanded Tregs typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of expanded Tregs and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

In some embodiments, expanded Tregs are encapsulated for administration, particularly where encapsulation enhances the effectiveness of the therapy, or provides advantages in handling and/or shelf life. Cells may be encapsulated by membranes, as well as capsules, prior to implantation. It is contemplated that any of the many methods of cell encapsulation available may be employed.

A wide variety of materials may be used in various embodiments for microencapsulation of expanded Tregs. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of expanded Tregs are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules). Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of cells.

Certain embodiments incorporate expanded Tregs into a polymer, such as a biopolymer or synthetic polymer. Examples of biopolymers include, but are not limited to, fibronectin, fibrin, fibrinogen, thrombin, collagen, and proteoglycans. Other factors, such as the cytokines discussed above, can also be incorporated into the polymer. In other embodiments of the invention, expanded Tregs may be incorporated in the interstices of a three-dimensional gel. A large polymer or gel, typically, will be surgically implanted. A polymer or gel that can be formulated in small enough particles or fibers can be administered by other common, more convenient, non-surgical routes.

The dosage of the expanded Tregs will vary within wide limits and will be fitted to the individual requirements in each particular case. The number of cells will vary depending on the weight and condition of the recipient, the number or frequency of administrations, and other variables known to those of skill in the art. The expanded Tregs can be administered by a route that is suitable for the tissue or organ. For example, they can be administered systemically, i.e., by intravenous administration, or can be targeted to a particular tissue or organ, such as the brain or spinal cord, by intrathecal administration.

The expanded Tregs can be suspended in an appropriate excipient in a concentration from about 0.01 to about 5×10⁶ cells/ml. Suitable excipients for injection solutions are those that are biologically and physiologically compatible with the cells and with the recipient, such as buffered saline solution or other suitable excipients. The composition for administration can be formulated, produced, and stored according to standard methods complying with proper sterility and stability.

### Dosing

Doses for humans or other mammals can be determined without undue experimentation by the skilled artisan, from this disclosure, the documents cited herein, and the knowledge in the art. The dose of expanded Tregs appropriate to be used in accordance with various embodiments of the invention will depend on numerous factors. The parameters that will determine optimal doses to be administered for primary and adjunctive therapy generally will include some or all of the following: the aberrant immune response or autoimmune disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the expanded Tregs are formulated, the way they are administered, and the degree to which the expanded Tregs will be localized at the target sites following administration.

In various embodiments, expanded Tregs may be administered in an initial dose, and thereafter maintained by further administration. Expanded Tregs may be administered by one method initially, and thereafter administered by the same method or one or more different methods. The levels can be maintained by the ongoing administration of the expanded Tregs. Various embodiments administer the expanded Tregs either initially or to maintain their level in the subject or both by intravenous injection. In a variety of embodiments, other forms of administration are used, dependent upon the subject's condition and other factors, discussed elsewhere herein.

Expanded Tregs may be administered in many frequencies over a wide range of times. Generally lengths of treatment will be proportional to the length of the disease process, the effectiveness of the therapies being applied, and the condition and response of the subject being treated.

### Uses

Administering the expanded Tregs is useful to reduce any of the overt symptoms of an aberrant immune response as described in this application. This may be based on underlying effects of the expanded Tregs, such as, prevention of autoimmune diseases by establishing and maintaining immunologic self-tolerance, induction of tolerance against dietary antigens, suppression of pathogen-induced immunopathology, regulation of the effector class of the immune response, suppression of T-cell activation triggered by weak stimuli, feedback control of the magnitude of the immune response by effector Th cells, protection of commensal bacteria from elimination by the immune system, and prevention of T cells that have been stimulated by their true high-affinity agonist ligand from killing cells that only express low-affinity T-cell receptor (TCR) ligands, such as the self peptide-major histocompatibility complex (MHC) molecule that positively selected the T cell.

The compositions of the present disclosure comprising expanded Tregs are useful for suppression of immune function in a patient. For example, as described above, autologous cells may be isolated, expanded and cultured in vitro as described herein, and subsequently administered or re-introduced to the patient. In some embodiments, such treatment is useful for example, to down-regulate harmful T cell responses to self and foreign antigens, and/or to induce long term tolerance.

In one example, the compositions and methods disclosed herein are directed to modulating an aberrant immune response in a subject, such as an autoimmune disorder or an allergy, by administering the expanded Treg compositions disclosed herein. In some embodiments, the subject is suffering from an autoimmune disorder or an allergic response, and the expanded Treg compositions are used to treat the autoimmune disorder or allergic disorder. In some embodiments, the subject is an animal model of an autoimmune disorder or allergic disorder. In other embodiments, the subject is a human afflicted with an autoimmune disorder or allergic disorder.

The expanded Treg compositions disclosed herein are used to treat, alleviate or ameliorate the symptoms, or suppress a wide variety of, aberrant immune responses, such as those described above.

In some embodiments, expanded Tregs disclosed herein are introduced into the subject to treat or modulate an autoimmune disorder or allergic disorder. For example, the subject may be afflicted with a disease characterized by having an ongoing or recurring autoimmune reaction or allergic reaction. In some embodiments, the modulating comprises inhibiting the autoimmune reaction or allergic reaction.

In some embodiments, expanded Tregs disclosed herein are administered to a subject for immunotherapy, such as, for example, in tumor surveillance, immunosuppression of cancers such as solid tumor cancers (e.g., lung cancer), and the suppression of in vivo alloresponses and autoimmune responses, including but not limited to, GVHD.

In some embodiments, the expanded Tregs disclosed herein may also be used to deliver suppressive or other biologic factors to sites of inflammation, such as, but not limited to IL-4, stem cell growth factors, and angiogenesis regulators. For example, in some embodiments, the expanded Tregs can be transduced with genes encoding a desired biological factor, which the cell will then produce once within the subject, e.g., at the site of inflammation.

In some embodiments, the expanded Treg compositions disclosed herein are indicated in infectious diseases in which the pathogenicity of the infection is not a result of the cytopathic effects of the pathogen, but rather the tissue damage caused by the immunoinflammatory response to the infectious agent. In diseases, such as hepatitis B or C or HSV-induced corneal inflammation, therapy with the expanded Tregs disclosed herein provides a unique opportunity to control viral-induced immunoinflammatory disease. Viruses, such as Coxsackie, are known to cause pancreatitis and have been associated with the development of Type 1 Diabetes. Thus, expanded Treg compositions as disclosed herein can be used to suppress local tissue damage caused by the infection and reduce the inflammation that incites autoimmune disorder development.

The subject methods find use in the treatment of a variety of different conditions and transplant situations in which the modulation of an aberrant immune response in a patient is desired. By way of example, but not by way of limitation, in the case of bone marrow or organ transplantation, composition comprising expanded Tregs disclosed herein may be administered during the time of surgery to prevent GVHD in a transplant patient. To keep the Tregs at the site until completion of the surgical procedure, in some embodiments, it is convenient to administer the Tregs in a pharmaceutically acceptable carrier, such as an artificial gel, or in clotted plasma, or by utilizing other controlled release mechanism known in the art.

In addition, other uses provided by the present invention include screening one or more agents or compounds for the ability to affect Treg expansion. Such a screening method includes (i) contacting certain cells (e.g., MAPC) with an agent or compound, (ii) contacting the cells with Tregs, and (iii) assessing the effect(s) of the agent or compound on the ability of the Tregs to expand. Assessment could be in vivo as in appropriate animal models.

A further use for the invention is the establishment of cell banks to provide Tregs for clinical administration. Cell bank construction can be done by expanding a population of Tregs as described herein, and then storing the expanded Tregs from that population for future administration to a subject.

It is also to be understood that expanded Tregs of the invention can be used not only for purposes of treatment, but also research purposes, both in vivo and in vitro to understand the mechanism(s) involved normally and in disease models. In one embodiment, assays, in vivo or in vitro, can be done in the presence of agents known to be involved in the biological process. The effect of those agents can then be assessed. These types of assays could also be used to screen for agents that have an effect on the events (e.g., inflammation) that are suppressed by the expanded Tregs of the invention. Accordingly, in one embodiment, one could screen for agents in a disease model that reverse the negative effects and/or promote positive effects. Conversely, one could screen for agents that have negative effects in a non-disease model.

All patents and scientific references cited herein are incorporated by reference for their teachings.

### NON-LIMITING EXAMPLE

### Example 1

Donor T198 Tregs (viable CD4+CD25highCD127low cells) were sorted from fresh PBMCs using GMP compliant antibodies and expanded using the current gold standard Treg protocol (anti-CD3/28 coated beads in the presence of IL-2 and rapamycin) in the presence or absence of 1:10 BM9 MultiStem. The current consensus ‘gold standard’ protocol for expansion of polyclonal Tregs involves FACS sorting or magnetic isolation of CD4+CD25highCD127low lymphocytes from peripheral blood followed by 4 rounds (each 10-14 days) of stimulation with anti-CD3, anti-CD28 beads in the presence of high concentrations of IL-2 and the immunosuppressive drug rapamycin (included to limit outgrowth of contaminating Teff cells).

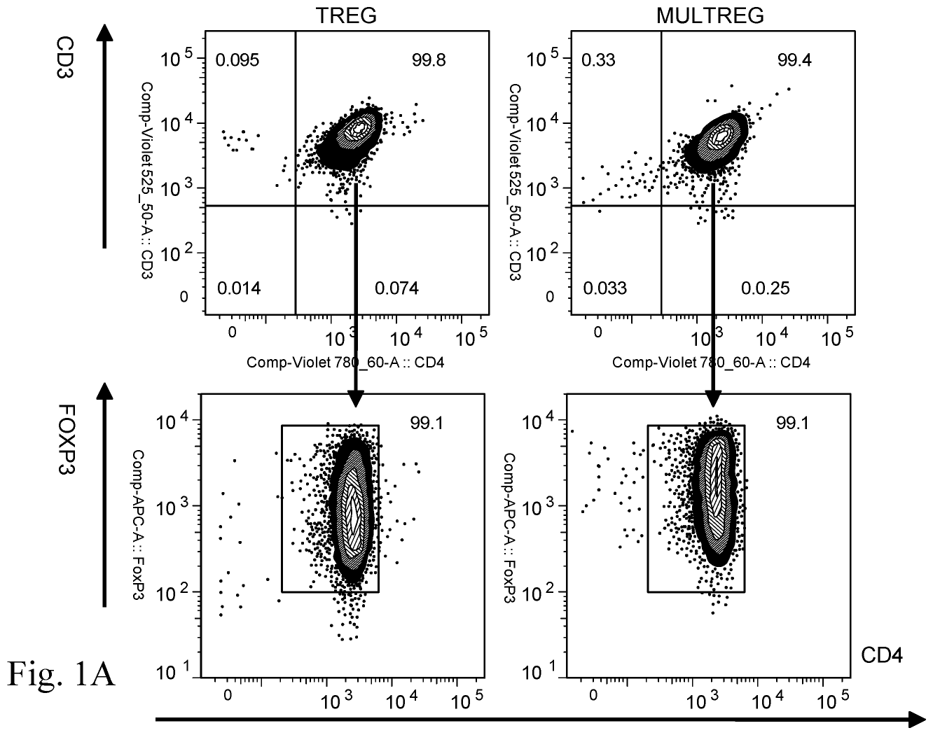

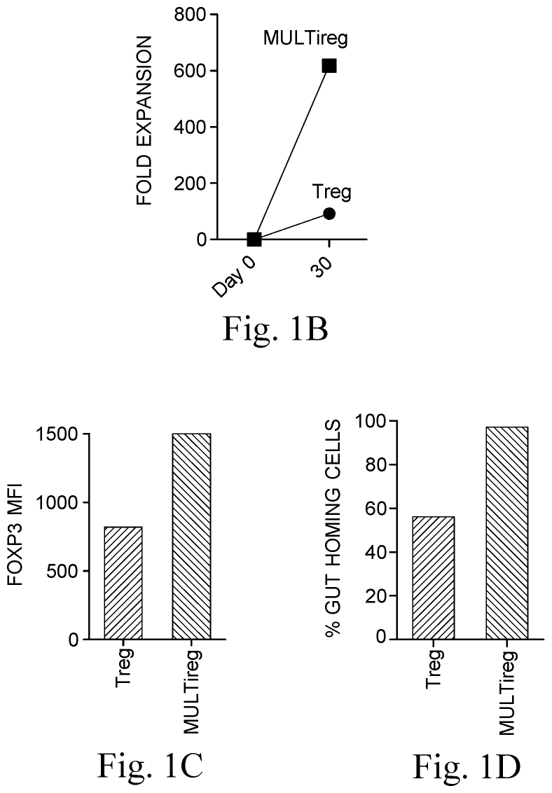

Cells expanded in the presence of MultiStem were given the concept name “MULTireg”. After 3 rounds of sequential (10 day) expansion, Tregs and MULTireg were cryopreserved or examined for total cell number, purity, FoxP3 expression, markers of differentiation (CCR7 and CD27), and tissue homing (CCR profile) by flow cytometry (FIGS. 1A-D).

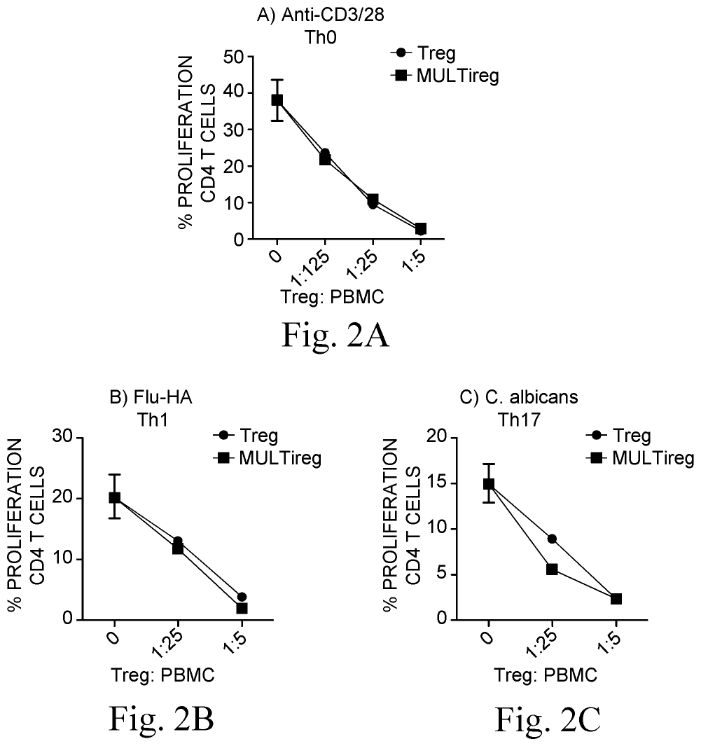

Cryopreserved MULTireg and Tregs were later thawed and assessed head to head for the ability to suppress T cell proliferation within traditional Treg suppression assays using autologous PBMCs activated with anti-CD3/28 beads. As an extension of this, we also assessed whether Tregs and MULTireg could suppress physiological antigen driven responses that require APC function and inherently provoke Th1 (Influenza Hemagglutinin) or Th17 (Candida albicans) effector cells known to cause graft rejection and autoimmunity (FIGS. 2A-C). In all experiments, Treg and Multireg demonstrated an equivalent level of suppression.

1. A method of enhancing proliferation of T regulatory cells (Tregs) in vitro, the method comprising contacting Tregs with cells (I), or conditioned medium from the cells (I), in the presence of one or more Treg stimulation agents, wherein the one or more Treg stimulation agents is present in an amount and for a time effective to stimulate proliferation of the Tregs, wherein the cells (I) are non-embryonic, non-germ cells that that have the ability to differentiate into cell types of at least two of endodermal, ectodermal, and mesodermal germ layers and/or express one or more of oct4, telomerase, rex-1 and rox-1, wherein the cells (I) are present in an amount and for a time effective to enhance proliferation of the Tregs.

2. The method of claim 1 wherein one or more Treg stimulation agents include soluble anti-CD3 antibodies, soluble anti-CD28 antibodies, anti-CD3/anti-CD28-coated beads and/or IL-2.

3. The method of claim 2 wherein one or more Treg stimulation agents include anti-CD3/anti-CD28-coated beads and IL-2.

4. The method of claim 1 wherein the one or more Treg stimulation agents include an antigen presenting cell.

5-8. (canceled)

9. The method of claim 1 further including contacting the Tregs and the cells (I) with an agent that suppresses T effector cell proliferation.

10. The method of claim 9 wherein the agent that suppresses T effector cell proliferation is rapamycin.

11. The method of claim 1 wherein the Tregs are expanded at least about 2-fold to about 1000-fold as compared to control Tregs, wherein the control Tregs are cultured under the same conditions but in the absence of cells (I).

12. The method of claim 11 wherein the Tregs are expanded at least about 5-fold to about 500-fold as compared to control Tregs, wherein the control Tregs are cultured under the same conditions but in the absence of cells (I).

13. The method of claim 12 wherein the Tregs are expanded at least about 6-fold as compared to control Tregs, wherein the control Tregs are cultured under the same conditions but in the absence of cells (I).

14-21. (canceled)

22. The method of claim 1 wherein the Tregs are CD4+CD14-CD25highCD127low.

23. The method of claim 1 wherein the Tregs, after proliferation, express a higher level of α4β7 as compared to control Tregs, wherein the control Tregs are cultured under the same conditions but in the absence of cells (I).

24-26. (canceled)

27. The method of claim 1 wherein the Tregs, after proliferation, express a lower level of CCR7 and CD27 as compared to control Tregs, wherein the control Tregs are cultured under the same conditions but in the absence of cells (I).

28-33. (canceled)

34. The method of claim 1 wherein the Tregs, after proliferation, express a higher level of FoxP3 as compared to control Tregs, wherein the control Tregs are cultured under the same conditions but in the absence of cells (I).

35-37. (canceled)

38. The method of claim 1 wherein the Tregs, after proliferation, have an increased potency to reduce T effector cell proliferation.

39-44. (canceled)

45. The method of claim 1 wherein the Tregs are derived from peripheral blood.

46. The method of claim 45 wherein the Tregs that are contacted with the cells (I) are in an unfractionated population of PBMCs.

48-49. (canceled)

50. The method of claim 1 wherein the cells (I) can differentiate into cell types of endodermal, ectodermal, and mesodermal germ layers.

51. The method of any one of claims 1 and 50 wherein the cells (I) can differentiate into cell types of at least two of endodermal, ectodermal, and mesodermal germ layers and express telomerase.

52. The method of any one of claims 1 and 50-51 wherein the cells (I) can differentiate into cell types of at least two of endodermal, ectodermal, and mesodermal germ layers and express oct4.

53. The method of claim 1 wherein the cells (I) can differentiate into cell types of endodermal, ectodermal, and mesodermal germ layers and express telomerase and oct4.

54. The method of claim 1 wherein the cells (I) are derived from bone marrow.

55. A method for immune modulation in a subject in need thereof, said method comprising administering to the subject a therapeutically effective amount of Tregs produced by the method of claim 1.

56. The method of claim 55 wherein the immune modulation is effective to treat an aberrant immune response.

57. The method of claim 56 wherein the aberrant immune response is an autoimmune disease selected from the group consisting of graft-versus-host disease, Type 1 diabetes, lupus, multiple sclerosis, asthma, sepsis and solid organ transplantation.

58. Tregs produced by the method of claim 1.

59. A composition comprising T regulatory cells (Tregs), cells (I), or conditioned medium from the cells (I), and one or more Treg stimulation agents, wherein the one or more Treg stimulation agents is present in an amount effective to stimulate proliferation of the Tregs, wherein the cells (I) are non-embryonic, non-germ cells that that have the ability to differentiate into cell types of at least two of endodermal, ectodermal, and mesodermal germ layers and/or express one or more of oct4, telomerase, rex-1 and rox-1, wherein the cells (I) are present in an amount effective to enhance proliferation of the Tregs.

60-104. (canceled)

105. The composition of claim 59 wherein the cells (I) can differentiate into cell types of endodermal, ectodermal, and mesodermal germ layers.

106. The composition of any one of claims 59 and 105 wherein the cells (I) can differentiate into cell types of at least two of endodermal, ectodermal, and mesodermal germ layers and express telomerase.

107. The composition of any one of claims 59 and 105-106 wherein the cells (I) can differentiate into cell types of at least two of endodermal, ectodermal, and mesodermal germ layers and express oct4.

108. The composition of claim 59 wherein the cells (I) can differentiate into cell types of endodermal, ectodermal, and mesodermal germ layers and express telomerase and oct4.

109. The composition of claim 59 wherein the cells (I) are derived from bone marrow.
